(19) United States
(12) Patent Application Publication
(10) Pub. No.: US 2007/0270741 A1
(43) Pub. Date: Nov. 22, 2007

(54) Transseptal needle assembly and methods

(76) Inventors: James A. Hassett, Eden Prairie, MN (US); Richard E. Stehr, Stillwater, MN (US)

Correspondence Address: SJM/AFD-WILEY, 14901 DEVEAU PLACE, MINNETONKA, MN, 55345-2126, US

(21) Appl. No.: 11/647,312

(22) Filed: Dec. 29, 2006

Related U.S. Application Data
(60) Provisional application No. 60/800,853, filed on May 17, 2006.

(51) Int. Cl. A61M 29/00

(52) U.S. Cl. 604/96.01

(57) ABSTRACT

The instant invention relates to transseptal access systems and methods for accessing the left atrium from the right atrium by crossing the interatrial septum. In particular, the instant invention is directed toward medical devices used with catheter assemblies in cardiology procedures that require transseptal puncture(s). The puncture assemblies have a moveable puncture device within a dilator. The puncture device is biased in a retracted position. The position of the puncture assembly is precisely locatable. The puncture assembly is preferably flexible along the majority of its length, and, therefore, can be used with any catheter assembly of any predetermined shape. The puncture device is adjustable from a position within the dilator to a position extending beyond the end of the dilator when necessary for use in transseptal procedures.

### CROSS-REFERENCE TO RELATED APPLICATIONS

This application claims priority to U.S. provisional application No. 60/800,853, filed 17 May 2006, which is hereby incorporated by reference as though fully set forth herein.

### BACKGROUND OF THE INVENTION

a. Field of the Invention

The instant invention relates to needle assemblies and methods for puncturing or piercing tissue within the body, including, for example, transseptal access systems and methods for accessing the left atrium from the right atrium by crossing the interatrial septum. In particular, embodiments of the instant invention are directed toward medical devices used with catheter assemblies in cardiology procedures that require transseptal puncture(s). The puncture assemblies have a moveable puncture device within a dilator being biased in a retracted position, the position of which is precisely locatable. The puncture assembly is preferably flexible along the majority of the length of the assembly, and therefore can be used with any catheter assembly of any predetermined shape, and adjustable from a predetermined position within the shaft to a position extending beyond the end of the dilator when necessary for use in transseptal procedures.

The puncture device is preferably axially flexible, but longitudinally rigid when placed within the lumen of the dilator. A safety mechanism, such as a spring biased member, a clip spacer, or similar locking mechanism, is located at the proximal end of the assembly, preferably within an operable handle providing for extension of the puncture assembly beyond the distal tip of the dilator, only upon the affirmative action of a user. When the force exerted by the user is removed, the puncture assembly automatically retracts back into the initial position within the dilator. Thus, when not being used for purposes of puncturing the septum, the puncture device is maintained within the dilator thereby increasing the safety of transseptal procedures.

b. Background Art

The human heart includes a right ventricle, a right atrium, left ventricle, and left atrium. The right atrium is in fluid communication with the superior vena cava and the inferior vena cava. The tricuspid valve separates the right atrium from the right ventricle. The right atrium is separated from the left atrium by a septum that includes a thin membrane known as the fossa ovalis.

A wide variety of diagnostic and therapeutic procedures have been developed in which a catheter is transluminally advanced within a guide sheath or over a guidewire into various chambers and across valves of the heart. The most difficult chamber of the heart to access with a catheter is the left atrium. Access to the left atrium through the pulmonary artery is not possible. Approaches from the left ventricle are difficult, may cause arrhythmias and may present difficulty in obtaining stable catheter positioning. Accordingly, one of the accepted methods of accessing the left atrium involves catheterization through the femoral or left subclavian vein into the right atrium, and subsequent penetration of the interatrial septum, the fossa ovalis, to gain entry to the left atrium. This procedure is commonly referred to as transseptal catheterization.

The objectives of left atrial access can be either diagnostic or therapeutic. One therapeutic use is electrophysiological intervention, e.g., left atrial ablation. Catheter ablation involves the placement of energy (typically RF) through a catheter, into various locations of the heart to eradicate inappropriate electrical pathways affecting the heart function. When these locations are in the left atrium, the catheter through which the RF energy is placed typically is itself placed through transseptal catheterization.

Despite clinical acceptance of a wide variety of procedures which require access to the left atrium, significant room for improvement remains in the actual access technique. A number of risks, in addition to the risks associated with normal heart catheterization, are inherent in transseptal catheterization. For example, a major risk present stems from the use of known transseptal devices, which typically have a puncture device, such as a needle and/or stylet, exposed externally from the dilator. The exposed nature of the puncture device renders adjustment of the assembly within the heart difficult, as it increases the risks of unanticipated puncture within the guide sheath during insertion, and detrimentally affects the maneuverability of the device to the appropriate point at the septum.

Known puncture assemblies typically have the distal portion of the puncture assembly exposed from the distal portion of the dilator. This configuration provides the puncture risks discussed above. Other assemblies with mechanisms to hold the puncture assembly within the dilator place a biasing mechanism at the distal tip of the assembly. Placing this mechanism at the distal tip presents additional problems with flexibility and maneuverability of the mechanism in operation. Thus, there is a need to provide a puncture assembly where the puncture device is safely maintained at a substantially fixed location within the dilator until the assembly is positioned at the puncture point of the septum, and further having a displacement mechanism located at the proximal end of the assembly. Such an improved structure greatly improves the overall functionality and safety of transseptal medical devices. Details of embodiments of this improved structure and related methods are described in more detail below.

### BRIEF SUMMARY OF THE INVENTION

The present invention provides for transseptal medical devices and methods having improved safety and maneuverability features.

According to one embodiment of the present invention, a transseptal medical device is provided comprising an elongate tubular member, such as a dilator, having a proximal end and a distal end, a puncture device disposed within the dilator, and a displacement mechanism operably connected to the puncture device at a proximal end of the puncture device, whereby the displacement mechanism is capable of advancing a distal portion of the puncture device from an initial position within the dilator to a position external to the dilator when a force is exerted upon the displacement mechanism. When the force is removed from the displacement mechanism, the distal portion of the puncture device retracts to the initial position within the dilator. Preferably, the displacement mechanism is operably connected to the puncture device or the dilator such that operation of the displacement mechanism moves the dilator in a direction toward a proximate end of displacement mechanism or moves the puncture device in a direction toward the distal end of the assembly.

The puncture device may have a length substantially equal to the length of the dilator, or substantially less than the length of the dilator yet still capable of being extended by the displacement mechanism to the position external to the dilator.

The puncture device may be comprised of a flexible polymer, a flexible metal, or any similar material known to those of skill in the art. The puncture assembly of the present invention may be flexible at a number of portions and may be comprised of a needle, or a curved needle. The puncture device may further include at least one rigid section located at the distal portion of the puncture assembly and/or at the proximal portion of the puncture device and may further have a flexible section intermediate the distal and proximate portions of the assembly.

The displacement mechanism of the medical device may further include a safety mechanism, such as a spring, a clip, or locking mechanism, operably connected to the puncture assembly. Preferably, the safety mechanism holds the puncture assembly within the dilator when the mechanism is in an unbiased, or locked position.

The dilator of the transseptal medical device may further comprise a dilator distal end and a dilator proximal end, the dilator distal end having a cross-sectional dimension smaller than a cross-sectional dimension of the dilator proximal end.

Embodiments of the present invention further contemplate methods for puncturing a septum of a patient's heart comprising the following steps: introducing a puncture assembly contained within a dilator into an area of the heart proximate a target area of the septum; extending the puncture assembly to a position external to said dilator proximate the target area of the septum; puncturing the target area of the septum; and retracting the puncture assembly to a position within the dilator. The methods may further comprise the step of advancing the dilator through the target area of the septum before retracting the puncture assembly, and may further comprise the step of advancing the dilator through the target area of the septum after retracting the puncture assembly.

Additional methods contemplated include methods for making an extendible transseptal medical device comprising the following steps: providing a dilator having an inner lumen; providing a puncture assembly having a puncture device and a flexible portion attached to the puncture device within the inner lumen of the dilator; and operably connecting a displacement mechanism to the puncture assembly allowing for the puncture assembly to be extended from a first position within the dilator to a second position external to a distal end of the dilator upon exertion of a force upon the displacement mechanism, and automatically retracting the puncture assembly to the first position when the force is removed from the displacement mechanism.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts a transseptal medical device according to one embodiment of the present invention having an outer sheath being configured for a transseptal puncture procedure, an elongate tubular member within the outer sheath, and a puncture device within the elongate tubular member. The medical device further contains a displacement mechanism at the proximal end of the device operably connected to the puncture device.

FIG. 2 depicts a transseptal medical device according to one embodiment of the present invention showing four components of the device: (1) a pre-formed sheath for use with transseptal procedures; (2) a flexible elongate tubular member; (3) a flexible puncture device; and (4) a displacement mechanism attached to the flexible puncture device.

FIG. 3 depicts a flexible puncture device according to one embodiment of the present invention connected to a displacement mechanism, both for use with the transseptal medical device of the present invention.

FIG. 4 depicts a displacement mechanism according to one embodiment of the present invention operably connected to a puncture device and further connected to a dilator having an inner lumen for housing the puncture device.

FIG. 5 depicts the distal end of a transseptal medical device according to one embodiment of the present invention in a puncture configuration having a sheath, a dilator, and a puncture device.

FIG. 6 is a cross-sectional view of a transseptal medical device according to one embodiment of the present invention having a dilator, a flexible puncture device disposed therein, and a displacement mechanism.

FIG. 7 is a cross-sectional view of a transseptal medical device according to another embodiment of the present invention having a dilator, a flexible puncture device disposed therein, and a displacement mechanism. The puncture device of this embodiment has a distal rigid portion, a proximal rigid portion, and a flexible needle section intermediate the distal and proximal rigid portions.

FIG. 8 is a schematic diagram exemplifying a transseptal puncture procedure wherein a transseptal medical device according to one embodiment of the present invention is inserted through the left subclavian vein, traveling into the right atrium.

### DETAILED DESCRIPTION OF THE INVENTION

In general, the instant invention relates to transseptal access systems and methods for accessing the left atrium from the right atrium by crossing the interatrial septum. In particular, the instant invention is directed toward medical devices used with catheter assemblies in cardiology procedures that require transseptal puncture(s).

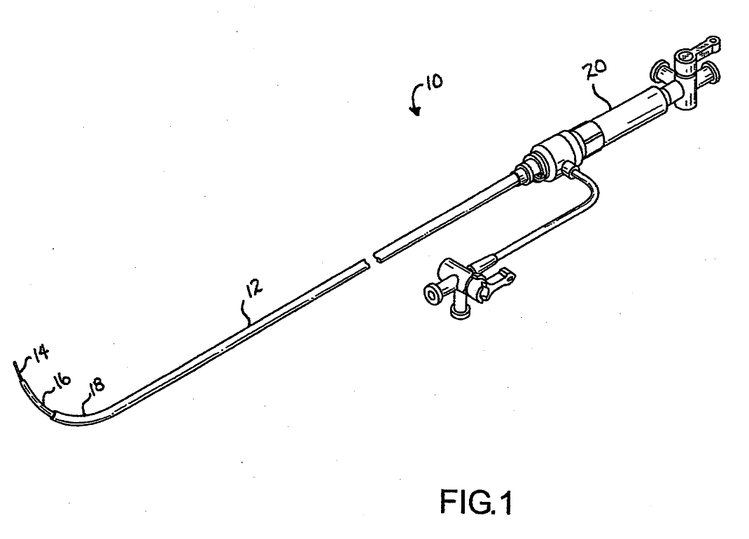

FIG. 1 shows an assembled puncture assembly 10 in accordance with one embodiment of the present invention, a portion of which is disposed within a sheath 12. The puncture assembly 10 has a retractable puncture device 14 disposed within a elongate tubular member, or for purposes of transseptal procedures, a dilator 16. The sheath 12 is preferably preconfigured with a bend 18 at an angle desirable for use with transseptal catheterization procedures. The puncture assembly 10 further includes a displacement mechanism 20 operably connected to either the puncture device 14 or the dilator 16, preferably the puncture device 14. In operation, the displacement mechanism 20 is operably connected to the puncture device 14 or the dilator 16 such that, when the displacement mechanism 20 is in an unbiased position, the distal end of the puncture device 14 is maintained at a predetermined, retracted position within the dilator 16 and the sheath 12. Upon exertion of a force upon the displacement mechanism 20, the puncture device 14 can be extended from an initial position within the dilator 16, to a distal position external to the dilator 16. This position is preferably the desired position for puncture of tissue in the body, e.g., the interatrial septum.

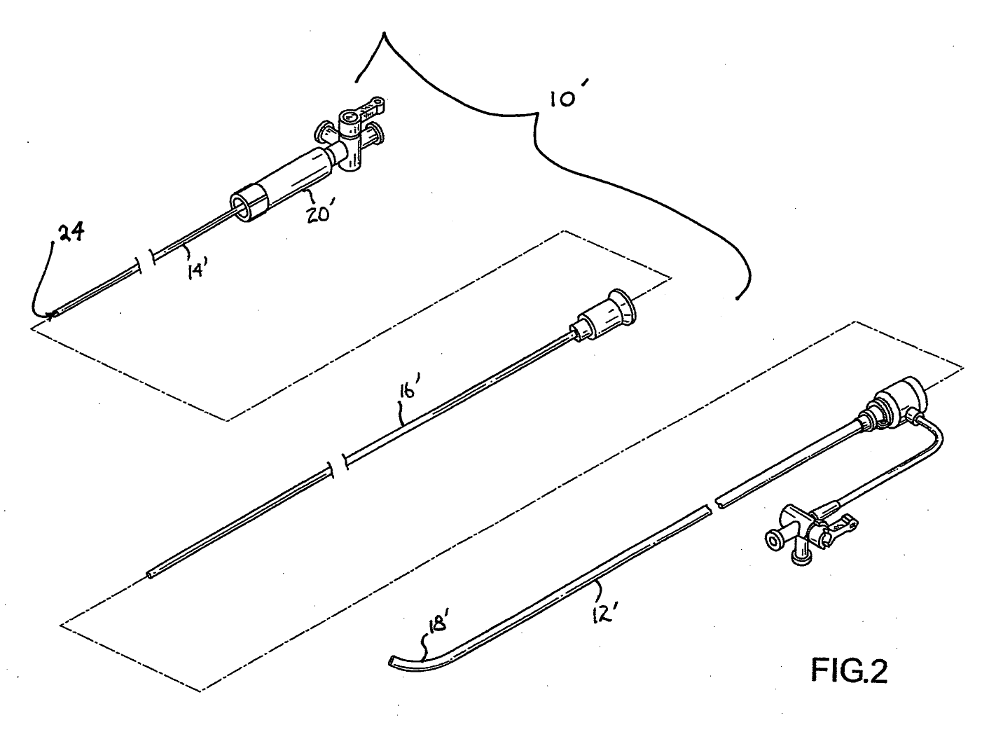

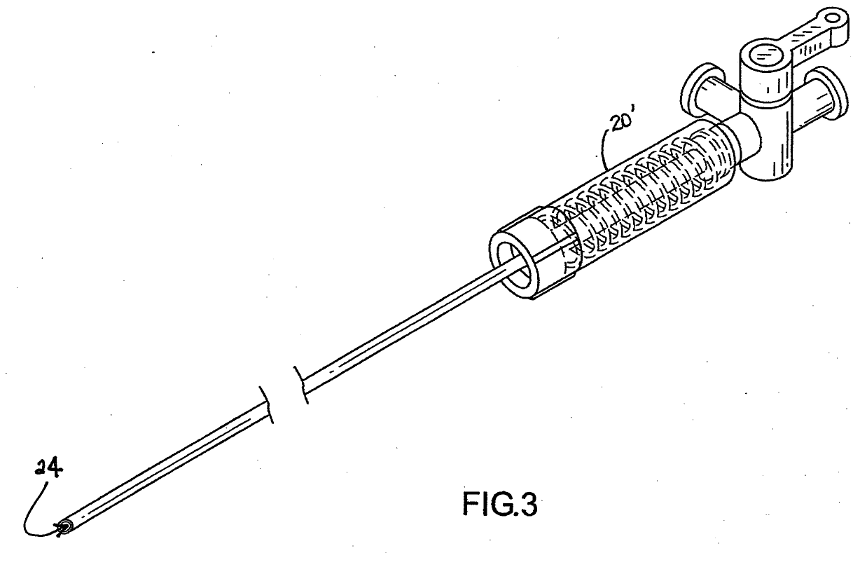

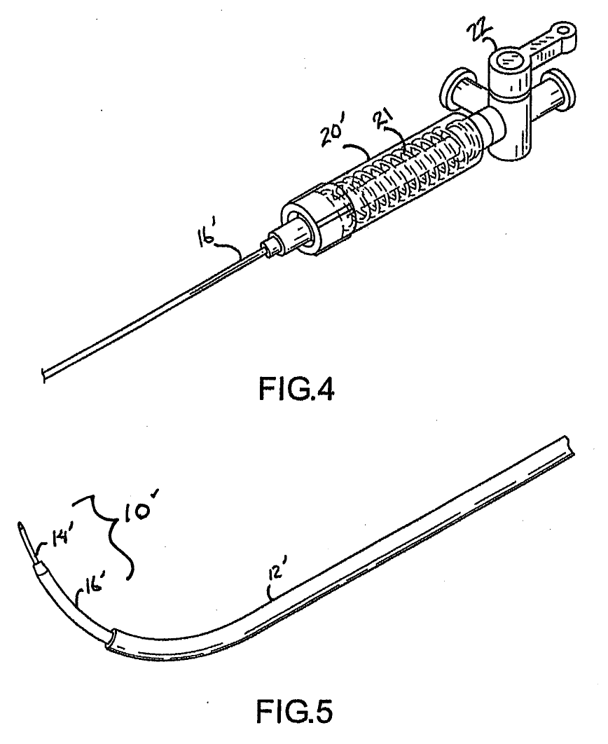

FIG. 2 shows various components of a flexible puncture assembly 10′ and a sheath 12′. The flexible puncture assembly 10′ includes a flexible puncture device 14′, a displacement mechanism 20′ operably connected to the puncture device 14′, and a dilator 16′. The sheath 12′ has a preformed bend 18′ at its distal end and is configured to house both the dilator 16′ and the puncture device 14′. FIG. 3 shows a flexible puncture device 14′, operably connected to a displacement mechanism 20′. FIG. 4 identifies a displacement mechanism 20′ operably connected to both a dilator 16′ and a flexible puncture device 14′ (not shown in FIG. 4) housed within the dilator and extending into the interior of the displacement mechanism 20′. A safety member or biasing member 21, e.g., a spring, a clip, or a locking mechanism, is disposed within the displacement mechanism 20′. Alternatively, a biasing mechanism 21 could be placed external to the distal end of the displacement mechanism 20′. Additionally, a valve 22 is operably connected to the puncture device 14′ such that fluids can be delivered to, or removed from a target site through a lumen 24 (FIGS. 2 and 3) within the flexible puncture device 14′. The valve 22 further allows for insertion and retraction of medical devices, such as an ablation electrodes for performance of desired medical procedures.

FIG. 5 shows a side view of a puncture assembly 10′, including a dilator 16′ and a puncture device 14′ disposed within a sheath 12′. The dilator 16′ extends a portion beyond the distal end of the sheath 12′. The puncture device 14′ is disposed within the dilator 16′. Preferably, the external diameter of the puncture device 14′ closely approximates the inner diameter of the dilator 16′ so as to provide axial rigidity to the puncture device 14′. This configuration allows the puncture device 14′ to be made of any flexible material, such as a polymers, plastics, or flexible metal constructions. This configuration allows for flexibility of the puncture device 14′ in axial or transverse directions to the longitudinal axis of the puncture device 14′, while simultaneously allowing for structural rigidity necessary along the longitudinal axis for advancing and retracting the puncture device 14′ within the dilator 16′.

FIG. 5 shows the puncture device 14′ in the extended position, i.e., the position effected by exertion of a force by a user upon the displacement mechanism 20′ (not shown in FIG. 5) operatively connected to the proximal end of the puncture assembly 10′. This position is preferable when the device is in position to pierce the tissue during a procedure. In the instance where there is no force acting on the displacement mechanism 20′, the puncture device 14′ is preferably at an initial, preset position within the dilator 16′. This preset, retracted position provides significant safety benefits over known puncture assemblies, where the puncture device remains exposed from the distal end of the dilator at virtually all times.

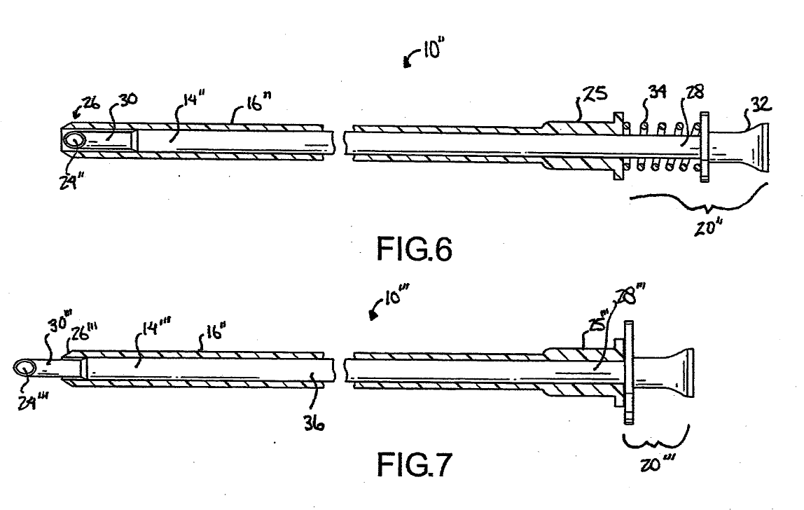

FIG. 6 is a cross-sectional view of a puncture assembly 10″ according to another embodiment of the present invention. The puncture assembly 10″ includes a dilator 16″ having a proximal end 25 and a distal end 26, the distal end 26 having a cross-sectional diameter less than the cross-sectional diameter of the proximal end 25. Disposed within an inner lumen of the dilator 16″ is a flexible puncture device 14″ having a proximal end 28 and a distal end 30. The distal end 30 of the flexible puncture device 14″ has a cross-sectional diameter less than the cross-sectional diameter of the proximal end 28. The flexible puncture device 14″ preferably has an inner lumen 24″ for receiving a stylet (not shown). The stylet is removable from the inner lumen. The inner lumen 24″ may also be used to deliver fluids to, or remove fluids from, a target site within the body.

The dilator 16″ is preferably comprised of a flexible material, such as biocompatible polymers, plastics, braided wire assemblies, and combinations thereof, or any other suitable material known to those of skill in the art. This flexible construction allows for the use of the puncture assembly 10″ with any known sheath (not shown) used for transseptal procedures.

The puncture device 14″ is preferably flexible along the majority of the length of the assembly 10″, and therefore can be used with any catheter assembly of any predetermined shape, and adjustable from a predetermined position within the shaft of the dilator 16″ to a position extending beyond the distal end 26 of the dilator 16″ when necessary for use in transseptal procedures. The puncture device 14″ can be made of any flexible material such as polymers, plastics, flexible metal coils, or any other flexible material known to those of skill in the art. The puncture device 14″ is preferably axially or transversally flexible, but longitudinally rigid when placed within the lumen of the dilator 16″. In this regard, the lumen of the dilator 16″ serves to contain the puncture device 14″ in a configuration that is amenable to exertion of a longitudinal force for purposes of extension or retraction while maintaining axial flexibility.

The puncture assembly 10″ further includes a displacement mechanism 20″ connected to the proximal end 25 of the dilator 16″ and/or the puncture device 14″. The displacement mechanism 20″ includes a proximal knob 32 for application of a force by a user. The displacement mechanism 20″ further includes a safety member or biasing member 34, shown as a spring, configured to hold the puncture device 14″ in an initial position where the distal end 30 of the puncture device 14″ is within the distal portion 26 of the dilator 16″. When a force is exerted on the displacement mechanism 20″, the distal end 30 of the puncture device 14″ extends a portion beyond the distal end 26 of the dilator 16″. When the force exerted by the user is removed, the puncture device 14″ automatically retracts back into the default biased position within the dilator 16″. Thus, when not being used for purposes of puncturing tissue, the puncture device 14″ is maintained within the dilator 16″ thereby increasing the safety of procedures, such as transseptal procedures.

While the biasing member 34 is shown as a spring, it is contemplated that other structures can also be used, such as a retractable clip, a locking mechanism, or a screw mechanism, to safely contain the distal end 30 of the puncture device 14″ at an initial position within the dilator 16″, until exertion of a force by a user. Preferably, the mechanism 20″ provides a restorative force to automatically retract the puncture device 14″ upon removal of a force acting upon the knob 32. It is contemplated, however, that this retraction could also be implemented by a manual exertion of a force on the knob 32 in a direction away from the distal portion 26. In this instance, the safety mechanism 20″ may be completely removed and/or replaced to prevent unwanted extension of the puncture device 14″ to a position external to the distal portion 26 of the dilator 16″.

FIG. 7 shows a cross-sectional view of an alternative embodiment of the puncture assembly 10′″ according to the present invention. In this embodiment, the puncture device 14′″ comprises a distal section 30′″, a proximal section 28′″, and an intermediate section 36. The distal 30′″ and proximal section 28′″ in this embodiment are preferably rigid sections with decreased flexibility. These sections may be made of any polymer, metal, or similar material known to those of skill in the art. The intermediate section 36 is preferably comprised of a flexible material such as polymers, plastics, flexible metal coils, or any other flexible material known to those of skill in the art. Preferably, the flexible intermediate section 36 tracks the curves of the dilator 16″ and/or sheath (not shown in FIG. 7). In this embodiment, the flexible intermediate section 36 provides for increased flexibility of the puncture device 14′″ during use, while the rigid sections 28′″ and 30′″ provide for increased strength at the proximal and distal ends of the puncture device 14′″ for applications where a more rigid puncture tip is desired, or where it is contemplated that additional force is necessary to pierce the targeted tissue area. This embodiment also provides increased ability to accurately locate and position the puncture assembly 14′″ for a more precise transseptal crossing. The portions may be bonded or attached together by any number of manners well known to one of ordinary skill in the art. While the embodiment shown in FIG. 7 identifies three separate portions of the puncture device 14′″, it is further contemplated that more or fewer portions are possible. For example, the puncture device 14′″ may have one portion at the distal end 30′″ made from a rigid material, while the remainder (28′″ and 36) is made from a flexible material. Similarly, it is contemplated that any combination of flexible and rigid portions may be provided depending on the desired combination of flexibility and rigidity of the transseptal device.

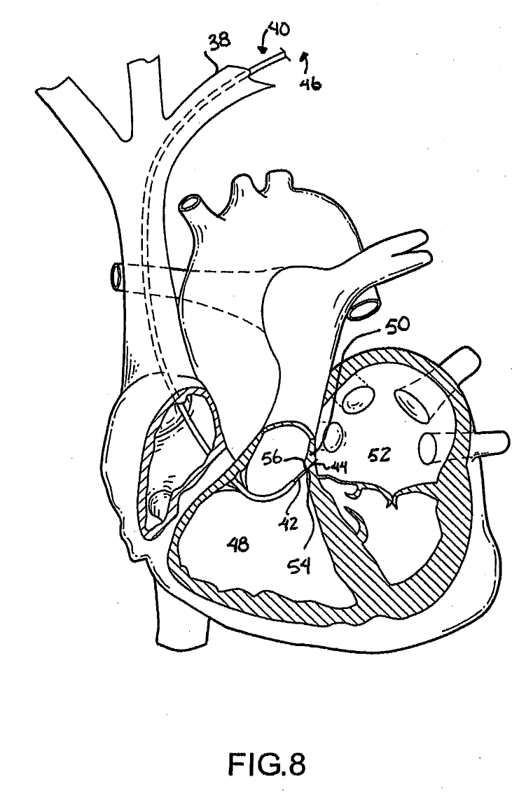

FIG. 8 shows a schematic diagram of a transseptal puncture procedure gaining access to the left atrium through the left subclavian vein 38. In this procedure, a transseptal medical device 40 according to one embodiment of the present invention is provided having a dilator 42, a puncture device 44, and a displacement mechanism (not shown in FIG. 8) located at the proximal end of the medical device 40. The medical device 40 is inserted through the left subclavian vein 38 and passed into the right atrium 48, where the device 40 is capable of performing a transseptal puncture allowing access to the left atrium 52 for further diagnostic or therapeutic treatment. The puncture device according to this embodiment comprises a flexible coil assembly (not shown) within the lumen of the dilator 42. The dilator 42 has a beveled distal end 54 for facilitation of the process. The puncture device 44 is shown in the extended position, i.e., with a force exerted upon the displacement mechanism. Thus, the puncture device 44 is in the position to puncture the interatrial septum 50.

In addition to the preferred embodiments discussed herein, the present invention contemplates methods for puncturing a septum of a patient's heart. The methods will be described in conjunction with the exemplary embodiment shown in FIG. 8. The methods preferably comprise the following steps: (1) introducing a puncture device 44 contained within a dilator 42 into an area of the heart proximate a target area 56 of the septum 50; (2) extending the puncture device 44 to a position external to the dilator 42 proximate the target area 56 of the septum 50; (3) puncturing the target area 56 of the septum 50; and (4) retracting the puncture device 44 to a position within the dilator 42. The methods further comprise the step of advancing the dilator 42 through the target area 56 of the septum 50 before retracting the puncture device 44, and optionally, advancing the dilator 42 through the target area 56 of the septum 50 after retracting the puncture device 44. In either instance, the result of the methods yield a conduit for delivery or removal of fluids or medical devices to any targeted area within the left atrium 52. This method therefore provides for safer access to the difficult to reach left atrium for the performance of medical procedures such as ablative, mappings, or other known procedures.

Additionally, the present invention contemplates methods for making an extendible transseptal medical device having increased safety features and beneficial maneuverability. The methods comprise the steps following: (1) providing a dilator having an inner lumen; (2) providing a puncture device having a flexible portion within the inner lumen of the dilator; and (3) operably connecting a displacement mechanism to the proximal end of the puncture device allowing for the puncture device to be extended from a first position within the dilator to a second position external to a distal end of the dilator upon exertion of a force upon the displacement mechanism. The method further contemplates automatically retracting the puncture device to the first position when the force is removed from the displacement mechanism.

Although a number of embodiments of this invention have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

1. A transseptal medical device comprising
a dilator having a proximal end and a distal end;
a puncture device disposed within the dilator; and
a displacement mechanism operably connected to the puncture device at a proximal end of the puncture device, whereby the displacement mechanism is capable of advancing a distal portion of the puncture device from an initial position within the dilator to a position external to the dilator when a force is exerted upon the displacement mechanism, and when the force is removed from the displacement mechanism, the distal portion of the puncture device retracts to the initial position within the dilator.

2. The transseptal medical device of claim 1, wherein the puncture device is a length substantially equal to a length of the dilator.

3. The transseptal medical device of claim 1, wherein the puncture device is a length substantially less than a length of the dilator yet still capable of being extended by the displacement mechanism to the position external to the dilator.

4. The transseptal medical device of claim 1, wherein the puncture device further comprises a distal end located proximate to the distal end of the dilator, and a flexible section.

5. The transseptal medical device of claim 4, wherein the puncture device comprises a needle.

6. The transseptal medical device of claim 4, wherein the puncture device comprises a curved needle.

7. The transseptal medical device of claim 4, wherein the puncture device further comprises a rigid section between the flexible section and the displacement mechanism.

8. The transseptal medical device of claim 1, wherein the dilator further comprises a dilator distal end and a dilator proximal end, the dilator distal end having a cross-sectional dimension smaller than a cross-sectional dimension of the dilator proximal end.

9. The transseptal medical device of claim 1, wherein at least a portion of the puncture device is further comprised of a flexible polymer.

10. The transseptal medical device of claim 1, wherein at least a portion of the puncture device is further comprised of a flexible metal.

11. The transseptal medical device of claim 1, wherein at least a portion of the puncture device is further comprised of a flexible coil of a polymer, a metal, or a combination thereof.

12. The transseptal medical device of claim 1, wherein the displacement mechanism is operably connected to the dilator, such that operation of the displacement mechanism moves the dilator in a direction toward a proximate end of the displacement mechanism.

13. The transseptal medical device of claim 1, wherein the displacement mechanism further comprises a spring mechanism operably connected to the puncture device.

14. The transseptal medical device of claim 13, wherein the spring mechanism holds the puncture assembly within the dilator when the spring mechanism is in an unbiased position.

15. A transseptal medical device comprising
an elongate tubular member having a proximal end and a distal end;
a puncture device disposed within the elongate tubular member; and
a displacement mechanism operably connected to the puncture device at a proximal end of the puncture device, whereby the displacement mechanism is capable of advancing a distal portion of the puncture device from an initial position within the elongate tubular member to a position external to the elongated tubular member when a force is exerted upon the displacement mechanism.

16. The transseptal medical device of claim 15 further comprising a mechanism to exert a retraction force on the displacement mechanism, thereby retracting the puncture device to the position internal the elongate tubular member.

17. A method for puncturing a septum of a heart comprising the following steps:
introducing a puncture device contained within a dilator into an area of the heart proximate a target area of the septum;
extending the puncture device to a position external to said dilator proximate the target area of the septum;
puncturing the target area of the septum; and
retracting the puncture device to a position within the dilator.

18. The method of claim 17 further comprising the step of advancing the dilator through the target area of the septum before retracting the puncture device.

19. The method of claim 17 further comprising the step of advancing the dilator through the target area of the septum after retracting the puncture device.

20. The method of claim 17, wherein the step of retracting the puncture device occurs automatically upon release of a force exerted by a user during the puncturing of the target area of the septum.

21. A method for making an extendible transseptal medical device comprising the following steps:
providing a dilator having an inner lumen;
providing a puncture device having a flexible portion within the inner lumen of the dilator;
operably connecting a displacement mechanism to the puncture device allowing for the puncture device to be extended from a first position within the dilator to a second position external to a distal end of the dilator upon exertion of a force upon the displacement mechanism, and automatically retracting the puncture assembly to the first position when the force is removed from the displacement mechanism.
